(19) United States
(12) Patent Application Publication
(10) Pub. No.: US 2014/0236194 A1
(43) Pub. Date: Aug. 21, 2014

(54) SPINAL DURAL REPAIR INSTRUMENTS AND METHODS FOR USING SAME

(71) Applicant: LMK RESEARCH, LLC, Mount Laurel, NJ (US)

(72) Inventors: Lawrence S. Deutsch, Moorestown, NJ (US); Mark A. Testauiti, Newtown Square, PA (US); Kamaldeep S. Momi, Medford, NJ (US)

(73) Assignee: LMK RESEARCH, LLC, Mount Laurel, NJ (US)

(21) Appl. No.: 13/767,901

(22) Filed: Feb. 15, 2013

(51) Int. Cl. A61B 17/04

(52) U.S. Cl. CPC A61B 17/0483
USPC 606/148

(57) ABSTRACT

Minimally invasive surgery for repairing a disruption in the spinal dura uses a pistol-grip forceps to permit the forceps to be used as a needle driver down a tubular cannula 14-20 mm in diameter and 40-120 mm long. The pistol grip permits one-handed use and provides an unobstructed view of the surgical site down the cannula. Jaws at the forceps' distal end are textured to frictionally engage the needle with sufficient force to enable the surgeon to drive the needle through tissue at the surgical site without damaging the needle. A bayoneted needle holder with a removable needle positioning tip having a unique configuration enables the surgeon to use the other hand to push a needle with an attached suture through the dura, after which the forceps are used to drive the needle and the suture through the edges of the disruption so the suture can be tied off.

### BACKGROUND OF THE INVENTION

1. Field of the Invention

The present invention relates to medical instruments and methods for using them, and more particularly, to instruments for use with minimally invasive spinal dural repair surgery and methods for performing surgical procedures using such instruments.

2. Description of Related Art

U.S. patent application Ser. No. 13/199,024 (“the '024 application”) by the present inventors describes a set of surgical instruments, and methods for using them, that are particularly adapted for repairing tears, incisions, or other disruptions of a patient's spinal dura mater. As the '024 application explains, the dura mater (or simply the “dura”) is the outermost of the three layers of the meninges surrounding the spinal cord and the brain. A primary function of the spinal dura is to protect, surround, and support the spinal cord. It forms the dural sac which extends between the foramen magnum and the coccyx and inside of which are the arachnoid mater, subarachnoid space, pia mater, and the spinal cord, nerves and roots. Disruptions in the spinal dura can cause leakage of cerebrospinal fluid from the dural sac, which can cause the pressure around the brain and spinal cord to drop and cause severe headaches, and possibly result in serious infection. Such infections can lead to meningitis and other symptoms, such as swelling of the brain. Although disruptions of the dura may heal themselves and not cause any symptoms, they often require surgical intervention.

Open field surgery for spinal procedures in general, and for dural repairs in particular, provides better access to the surgical site but it can lead to complications and longer healing times, and can also require more physical therapy until the patient is fully recovered. However, even with open field surgery, spinal dural repairs can be difficult to perform because the surface of the spinal dura is accessible only through the spaces between vertebrae. A proven successful minimally invasive surgical technique that provides access to a patient's spinal region is described in U.S. Pat. No. 5,792,044, the entire disclosure of which is incorporated herein by reference. In this technique, illustrated in FIG. 10 of the patent and described beginning at column 10, a guidewire is inserted though an incision at a medial posterior approach to the patient's spine. A series of larger and larger tissue dilators are placed one after the other, first over the guidewire and then over each previously placed dilator, the last dilator of which will typically have a diameter of between about 13 mm to about 25 mm. A tubular retractor with a cannula diameter of between about 14 mm and 26 mm slides over the dilators and into the patient's spinal region, after which the dilators and guidewire are removed. The tubular retractor, which is anchored to surrounding structure (such as the surgical table), defines a surgical field at the open bottom of the retractor cannula. The tubular retractor can be between 40 mm and 120 mm in length, depending on the physical characteristics of the patient. For example, longer tubes will be required to reach the spinal region of obese patients. A number of surgical procedures that can be performed by accessing the spinal region in this fashion are described in Minimal Access Spinal Technologies™, brochure of Medtronic Sofamor Danek USA, Inc., Memphis, Tenn. (2004) (“the Medtronic brochure” the contents of which are incorporated herein by reference.

It is important to understand that the only access to the surgical site for these surgeries is through the tubular retractor, which requires manipulating one, two, or more instruments at a time from outside the retractor with the only line of sight to the surgical field being down the retractor cannula. The Medtronic brochure illustrates a number of so-called “bayoneted” instruments with offset handles that can be used to access the surgical site down the retractor cannula. The utility of bayoneted instruments to access areas through a tubular cannula is well illustrated in U.S. Pat. No. 6,962,582. The prior art also includes microsurgical bayoneted needle drivers, but known instruments of this nature use a pinching mechanism that is bulky and difficult to maneuver at the distal end of a tubular cannula as small as 14 mm and as deep as 120 mm.

Examples of some known suturing instruments and techniques suitable for use in open field surgery are shown in U.S. Pat. No. 1,037,864, No. 2,370,545, No. 4,161,951, No. 5,730,747, and No. 7,208,004, and in Pubs. No. US 2005/0090841 and No. US 2009/0005795. However, it will be immediately apparent to those skilled in the art that none of these instruments or their like are well suited for suturing the spinal dura, and would be especially difficult or even impossible to use for that purpose at the distal end of a tubular retractor like that used in the surgical techniques described in the Medtronic brochure. U.S. Pat. No. 2,370,545 and Pub. No. US 2005/0090841 are non-bayoneted suturing instruments and would not be suitable for driving a needle through the dura at the distal end of such a tubular retractor, because the surgeon would have no line of sight to the surgical field where the suturing is to be performed. Moreover, the stitching instrument in U.S. Pat. No. 2,370,545 has a bulky distal end with protruding surfaces and edges that would be practically impossible to use down a tubular retractor in the type of surgery discussed here and would risk damaging the fragile spinal dura in any case. The suturing devices in U.S. Pat. No. 7,208,004 and Pub. No. 2009/0005795 are straight tweezer-like devices that would not permit the surgeon to see down the tubular retractor to the surgical site.

Complicating matters is the fact that the spinal dura is a soft, thin, and in some cases very delicate membrane that can be easily torn, especially in older patients. Driving a suturing needle through the dura at the bottom of the tubular retractors used in minimally invasive surgical techniques like that described above, and being able to manipulate the suture to complete the repair and tie off the suture, has heretofore been extremely difficult using existing surgical implements. For example, the above-mentioned U.S. Pat. No. 1,037,864, No. 4,161,951, and No. 5,730,747 disclose suture-passing implements with pivoting needle driving and needle receiving jaws. The implements in U.S. Pat. No. 1,037,864 and No. 4,161,951 are constructed like pliers with jaws that open and close, making these implements practically impossible to use at the bottom of a long, narrow cannula. And although the forceps U.S. Pat. No. 5,730,747 has a pistol grip with jaws at the forceps' distal end, which theoretically would afford the surgeon a line of sight down a cannula, the jaws would still obscure from sight the surgical field at the end of the cannula. And the instruments in all of these patents are inherently unsuitable for dural repairs because the jaws would risk damaging surrounding tissue and/or small neurofibrils that can float into the rent in the dura. Moreover, the configuration of these types of devices requires them to be positioned perpendicularly to the rent in the dural tissue, which immediately compromises their utility in a minimally invasive surgical procedure at a site accessible only through a long, narrow tubular cannula.

Although the Medtronic brochure describes a number of surgical procedures available using the minimally invasive techniques described therein (and in U.S. Pat. No. 5,792,044), our co-pending '024 application describes the first known instruments suitable for employing those techniques for spinal dural procedures such as repairing tears. While our previous instruments are very effective for their purpose, we have discovered certain improvements that will provide even more effective instruments with greater utility when used in this type of minimally invasive surgery.

### SUMMARY OF THE INVENTION

It is an object of the present invention to provide instruments in addition to those described in our co-pending U.S. patent application Ser. No. 13/199,024 for performing surgical procedures on the spinal dura, particularly for making dural repairs by suturing the dura at the distal end of small diameter tubular retractors such as those used in the type of minimally invasive techniques already described.

In accordance with that and other objects, the invention in one aspect comprises a needle driving instrument in the form of pistol-grip forceps with jaws at a distal end that are configured in a manner enabling them to grasp a suturing needle with sufficient force to perform a suturing operation on the spinal dura at the distal end of a small diameter tubular retractor, but without damaging the needle and at the same time reducing the risk of trauma to the spinal dura and surrounding/associated tissue.

In another aspect of the invention, a bayoneted surgical needle pushing instrument facilitates surgical procedures involving this type of minimally invasive technique by incorporating a bayoneted holder having at a distal end thereof a needle positioning tip with a unique configuration that securely holds a surgical needle in an optimum orientation for enabling a surgeon to manipulate it into position for pushing the needle through the spinal dura at the distal end of a small diameter tubular retractor.

In yet another aspect of the invention, the needle positioning tip can be removed from the pushing instrument, which enables it to he separately sterilized before attachment to the pushing instrument, removed and discarded after use, and then replaced with another sterile positioning tip, all during the same surgical procedure. In a related aspect of the invention, a plurality of sterilized needle positioning tips with premounted surgical needles are provided in a cassette that the surgeon can access during a surgical procedure. In a more specific embodiment, the needle positioning tip is deformable to permit the surgeon to bend it into different configurations in order to facilitate the repair by adapting the configuration to the location/orientation of tissue in the vicinity of the surgical site.

A method aspect of the invention involves using a needle driving instrument and needle pushing instrument in accordance with the above described aspects of the invention to perform surgical procedures such as repairs to the spinal dura at a surgical field defined at the bottom distal end of the cannula of a tubular retractor. In a more specific embodiment, the tubular cannula is between 14 mm and 26 mm in diameter and between 40 mm and 120 mm in length.

This Summary is provided to introduce a selection of concepts in a simplified form that are further described below in the Detailed Description. This Summary is not intended necessarily to identify key or essential features of the claimed subject matter, nor is it intended to be used in determining the scope of the claimed subject matter.

### BRIEF DESCRIPTION OF THE DRAWINGS

The objects of the invention will be better understood from the detailed description of its preferred embodiments which follows below, when taken in conjunction with the accompanying drawings, in which like numerals and letters refer to like features throughout. The following is a brief identification of the drawing figures used in the accompanying detailed description.

FIG. 1 is a side view of a pistol-grip forceps with jaws configured for providing a needle driving instrument according to an embodiment of the present invention.

FIG. 2 is a side view of the forceps in FIG. 1 with the jaws in an open position.

FIG. 3 is an isometric view of the distal end of the forceps shown in FIGS. 1 and 2, enlarged to illustrate details of the jaws thereof.

FIG. 4 is a side view of a needle pushing instrument comprising a bayoneted needle holder and a removable needle positioning tip according to an embodiment of the present invention.

FIG. 5 is an exploded isometric view of the distal end of the needle pushing instrument shown in FIG. 4, enlarged to illustrate details of the connection between the needle holder and the removable needle positioning tip and of a suturing needle suitable for dural repairs.

FIG. 6 is an isometric view of the distal end of the needle pushing instrument in FIG. 5 with the needle positioning tip mounted to the needle holder and the needle in place on the needle positioning tip.

FIG. 7 is an isometric view of a cassette holding a plurality of needle assemblies comprising needle positioning tips and premounted needles suitable for use with the needle pushing instrument shown in FIGS. 5 and 6.

FIG. 8 illustrates the manner in which the forceps and needle holder shown in FIGS. 1 to 6 provide a line of sight for a surgeon performing a dural repair using a minimally invasive surgical technique in which access to the surgical field is through the cannula of a tubular retractor; FIG. 8A is an isometric view of the tubular retractor shown in FIG. 8.

FIGS. 9 to 11 illustrate a method of repairing a dural tear using the surgical instruments described herein in a minimally invasive surgical procedure.

One skilled in the art will readily understand that the drawings are not strictly to scale, but nevertheless will find them sufficient, when taken with the detailed descriptions of preferred embodiments that follow, to make and use the present invention.

### DETAILED DESCRIPTION OF PREFERRED EMBODIMENTS

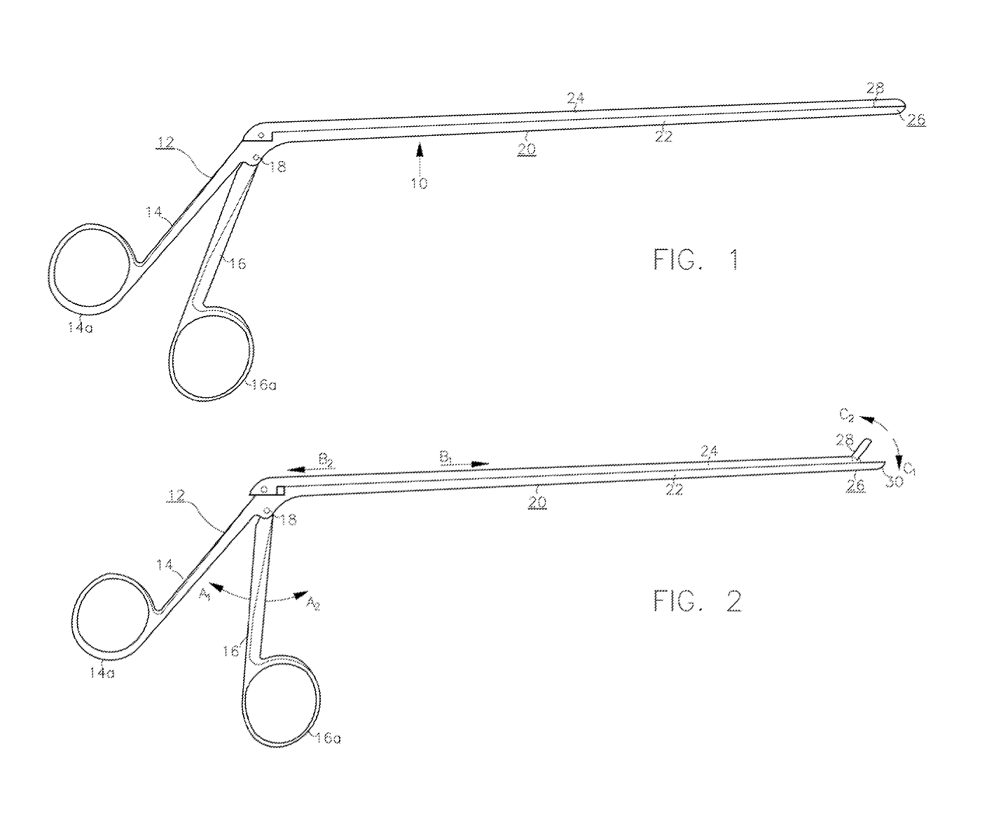

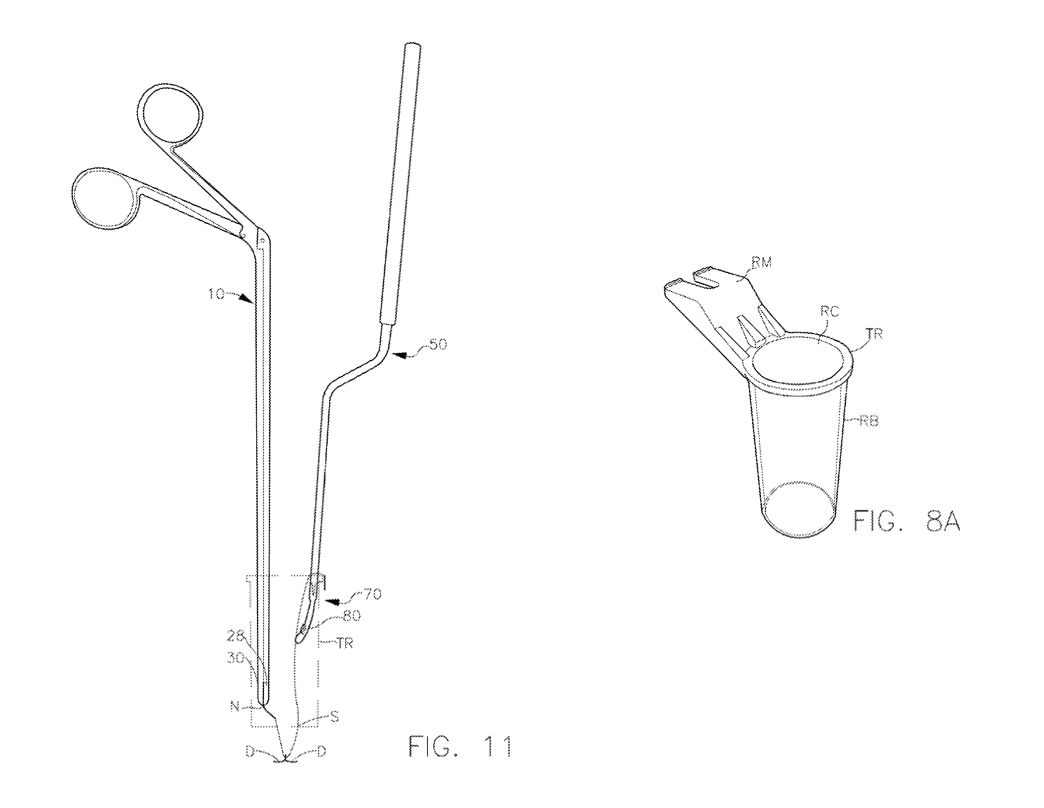

Novel surgical instruments and accessories embodying certain aspects of the invention will be described in connection with the particular embodiments shown in FIGS. 1 to 7. Thereafter, surgical procedures using such instruments in accordance with method aspects of the invention will be described with reference to FIGS. 8 to 11. As the following description of exemplary embodiments of the claimed subject matter proceeds, terms indicating direction or orientation, such as “up,” “down,” “upper,” “lower,” “top,” “bottom,” etc., may be used to facilitate the description of these embodiments. They do not imply that the claimed subject matter is limited to a particular orientation of the component being described.

Improved Instruments for Spinal Dural Surgical Procedures

With reference first to FIGS. 1 to 3, a needle driving instrument in the form of a forceps 10 has a proximal end 12 with a pair of pistol-grip handles that include an anchoring handle 14 and an operable handle 16 mounted for rotation relative to one another at a pivot point 18. The handles include a grip 14a for a surgeon's thumb and a grip 16a for his or her finger, thus permitting one-handed operation. It will be understood that the instrument is not limited to use in such fashion, and that in or during a given procedure the surgeon could use the grip 16a for his or her thumb and the grip 14a for a finger.) A shaft portion 20 includes a lower anchoring member 22 and an upper operable member 24 that is mounted to the anchoring member 22 for longitudinal sliding movement relative thereto in a manner that will be described in a moment. As the drawings show, the handles 14 and 16 are mounted in lateral offset relation to, the shaft portion 20, thus providing the “pistol grip” configuration that characterizes this type of instrument. The forceps 10 terminates at a distal end 26 with a jaw mechanism. The jaw mechanism comprises a movable jaw 28 and a fixed jaw 30, the latter being an integral part of the anchoring member 22 of the shaft portion 20. The forceps 10 thus far described has a conventional construction, and as known to those skilled in the art, is operated by moving the anchoring handle 14 and the operable handle 16 and toward and away from each other.

To close the movable jaw 28, the operable handle 16 is squeezed toward the anchoring handle 14 in the direction of the arrow A₁ in FIG. 2, to rotate the handle 16 about pivot point 18 relative to the handle 14. The operable member 24 of the shaft portion 20 is connected to the anchor member 22 by an internal mechanism (not shown) that causes the operable member 24 to slide longitudinally relative to the anchor member 22 in the direction of arrow B₁ when the handles are squeezed together. In turn, another internal mechanism (not shown) between the anchor member 22 and the operable member 24 causes the movable jaw 28 to rotate in the direction of arrow C₁ to move it to its closed position (FIG. 1). Conversely, to open the movable jaw 28, the surgeon moves the operable handle 16 in the direction of arrow A₂, causing the operable member 24 of the shaft portion 20 to slide in the direction of arrow B₂, which in turn rotates the movable jaw 28 in the direction of arrow C₂ to its open position (FIG. 2).

Conventional pistol-grip forceps having the construction described above have long been used to perform surgical procedures in locations that would otherwise be inaccessible to the surgeon. Examples of known types of pistol-grip forceps include pituitary rongeurs, which are cutting devices, and toothed grasping forceps, which are used to hold tissue. No known types of pistol-grip forceps had been proposed for use as needle driving instruments until our co-pending '024 application. Nor would theretofore known forceps be suitable for that purpose because their mating jaw surfaces typically had sharp edges for cutting tissue samples or coarse teeth for securely grasping slippery tissue, and thus would not be able to hold a fine surgical needle with sufficient force to pull it through tissue without damaging the needle. In addition, sharp and/or coarse jaw surfaces of known pistol-grip forceps would present a risk of damaging the spinal dura and neighboring/associated tissue, such as small neurofibrils that can float into the rent in the dura that is being repaired.

Thus, in accordance with one aspect of the invention, the otherwise conventional forceps 10 is specially adapted for use as a needle driving instrument. To that end the mating jaw surfaces of the forceps are textured to enhance the grip on the needle, but the surfaces are nevertheless configured so that they do not damage the needle or tissues that might be present in the vicinity of the rent in the dura. In that regard, needles used for suturing the spinal dura are generally between 0.6 mm and 0.7 mm in diameter (see needle N in FIG. 5). FIG. 3, which shows the movable jaw 28 in the open position, is a detail of the distal end 26 of the forceps 10 showing the jaws 28 and 30 with mating jaw surfaces 28a and 30a that are configured to enable use of the forceps 10 to grasp these fine surgical needles firmly enough to drive them through spinal dural tissue without damaging the needle. A substantial portion of these surfaces is knurled with small raised ridges (Which may or may not crisscross) and/or beads that ensure a firm grip on a suturing needle when the surgeon squeezes the handles 14 and 16 together as described above. To prevent damage to the needle (and to neighboring tissue) the ridges and/or beads will present raised protuberances that preferably have generally rounded, or otherwise blunted, top. ends which are preferably spaced apart a distance about the same order of magnitude as the diameter of the needle, which in this instance with the needle described above, will be preferably between 0.5 mm to 1.0 mm. Other surface treatments, such as etching to provide a matte finish, are also within the scope of the invention, the general concept being that the jaws have mating textured surfaces for enhancing frictional engagement with a surgical needle, while minimizing the prospect of damaging the needle or risking trauma to adjoining tissue when the surgeon uses the forceps in surgical procedures according to the methods described further below.

With reference next to FIGS. 4 to 6, as needle pushing instrument in the form of a bayoneted needle holder 50 that comprises a proximal handle portion 52 connected by an intermediate portion 54 to a distal needle holding portion 56. The proximal portion 52 comprises a handle for a surgeon performing a surgical procedure described further below. The proximal portion 52 lies generally along a first axis 52a and the distal portion 56 lies generally along a second axis 56a offset laterally from the first axis by a distance OS. This provides the needle holder 50 with a distinctive bayonet shape that is familiar to surgeons. Any of the shapes of known bayoneted instruments, such as those depicted in the Medtronic brochure and in patents such as U.S. Pat. No. 7,163,532, can be used as the basis of the needle holder 50, and all of the examples of such instruments depicted and described in that brochure and patent are incorporated herein by reference. The distal, intermediate, and proximal portions usually lie in a plane and are commonly formed integrally as an single unitary part from a suitable material, typically stainless steel. to facilitate sterilization, although other constructions and materials are possible. For example, a needle holder 50 intended to be discarded after a single procedure could be a plastic material if it is made sufficiently rigid for the purposes to be described. The needle holder could also be made of a deformable material that permits the surgeon to bend it into different shapes to facilitate the surgical procedure for which it is being used. The proximal portion 52 may have an enlarged diameter with a suitably knurled or otherwise textured surface to provide the surgeon with a better grip.

The distal end of the needle holder 50 has attached thereto a removable needle positioning tip 70 that represents an embodiment of another aspect of the invention. FIGS. 5 and 6 illustrate details of the needle positioning tip 70 and the manner in which it attaches to the needle holder 50. FIG. 5 includes a depiction of the cooperating distal end 58 of the needle holder 50 where the needle positioning tip 70 is mounted. The distal end 58 includes an axial indexing tab 60 extending therefrom and having first and second indexing pins 62 and 64 that extend in turn from the distal edge of the indexing tab 60. The needle positioning tip 70 at its proximal end has a transverse slot 72, which accepts the indexing tab 60, and two cooperating recesses (not shown) at the base of the transverse slot 72 which accept respective indexing pins 62 and 64. The indexing tab 60 tapers slightly in thickness toward the distal end thereof and the width of the transverse slot 72 narrows from its opening to its end in matching relation to the taper of the tab 60.

These mating parts of the needle holder 50 and the positioning tip 70 form a positioning means for positioning the needle positioning tip in a predetermined orientation on the needle holder. The invention of course includes any equivalent structure that will ensure the desired orientation of the positioning tip 70 on the needle holder 50. Moreover, the cooperating tapering of the tab thickness and the slot width facilitate initial registration of the tab and the slot before the positioning tip 70 is pushed home in the direction of the arrow in FIG. 5. It will be appreciated that the needle positioning tip 70 is small, on the order of 3 mm wide and 25 mm long, and mounting it on the needle holder during a surgical procedure could prove difficult without the assistance afforded by the tapering the indexing tab 60 and the transverse slot 72. In addition, the slot can optionally be made to taper at a lesser angle than the indexing tab, which will cause the tab to be wedged in place when it is fully home in the slot. The pins 62 and 64 can also be made to provide a slight interference fit with the cooperating recesses in the base of the slot. These provisions will help ensure that the needle positioning tip 70 does not become dislodged in use.

The needle positioning tip 70 further includes a flat main portion 71 between a rounded proximal portion 73 and a curved distal end portion 74, which is bifurcated to provide two fingers 75 and 76 that form a straight needle slot 78 therebetween. For most applications, the preferred orientation of the mounted positioning tip 70 is with the curved distal end portion 74 extending in a direction away from the handle portion 52 of the needle holder 50, as best seen in FIG. 4. Further, the flat main portion 71 will typically be perpendicular to the plane of the needle holder 50, and the needle slot 78 will be generally aligned with the axis 56a of the distal end 56 of the needle holder 50. However, it should be understood that the positioning tip can be made so that the needle positioning tip has other orientations for particular surgical procedures where it would better serve the purposes described herein. The top surface of the needle positioning tip main portion 71 (as depicted in FIGS. 5 and 6) carries a needle receptacle 80 with a straight bore 82 that is generally aligned with the needle slot 78. The opposite “bottom” surface of the needle positioning tip is smooth to reduce the risk of damaging tissue in the vicinity of the surgical site during the surgical procedure described below. For the same reason, the distal end of the needle positioning tip 70 is blunt with rounded edges. A further feature of this embodiment resides in constructing the indexing tab 60 and the pins 62 and 64 on the needle holder 50, and the transverse slot 72 and the cooperating recesses (not shown) in the positioning tip 70, so that they are symmetrical in the longitudinal and transverse directions. This permits the needle positioning tip 70 to be mounted on the needle holder 50 “upside down” from the position shown in FIG. 4 (that is, with the curved distal end portion 73 extending in the same direction as the handle portion 52 of the needle holder 50).

FIGS. 5 and 6 taken together illustrate the manner in which the positioning tip 70 releasably accepts a curved surgical needle N of the type typically used for suturing disruptions or tears in the spinal dura. FIG. 5 depicts the needle N separated from the positioning tip to better show the curved needle shaft portion NC and the needle's straight shank portion NS. A surgical suture S of suitable composition and gauge is permanently attached to the end of the needle at the needle shank. FIG. 6 shows the needle N in place and ready for use in surgical procedures discussed in detail below. The needle N is held in the proper position for performing such procedures by cooperation of the needle receptacle 80, which accepts the straight needle shank portion NS, and the needle slot 78, which captures the curved needle shaft portion NC. The suture S is folded over the needle shank portion NS as shown in FIG. 6 and the needle shank portion NS is inserted into the bore 82, which is dimensioned to provide a slight resistance to axial movement of the needle N when in the bore. Likewise, the fingers 75 and 76 have sufficient resilience to permit them to separate slightly when the needle shaft portion NC is pressed into the needle slot 78 as shown in FIG. 6. The needle slot 78 and the needle receptacle 80 are thus configured and dimensioned so that they cooperate to hold the needle N securely enough to enable it to be pushed through tissue, particularly the spinal dura in a preferred application, but allow the needle to be readily separated from the needle positioning tip 70 when desired. The needle N used to exemplify this aspect of the invention typically lies in a plane, but the invention encompasses needle positioning tips with needle slots and needle receptacles that will accept other needle configurations in accordance with the principles of implementation of the invention as described below with reference to FIGS. 8 to 11.

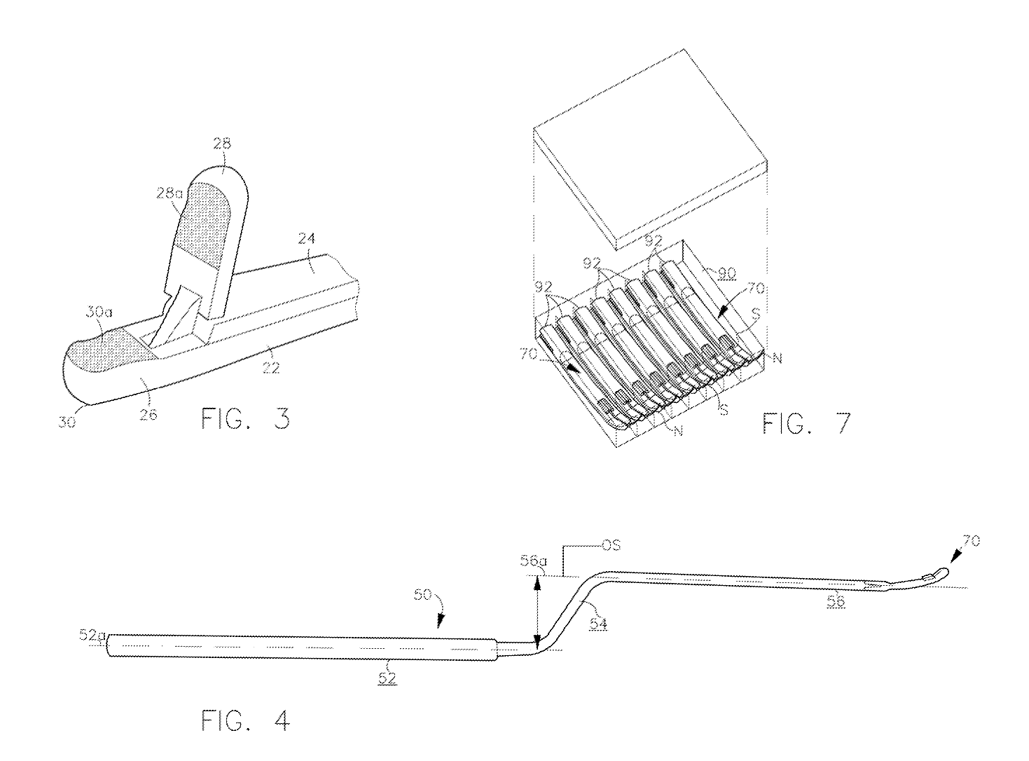

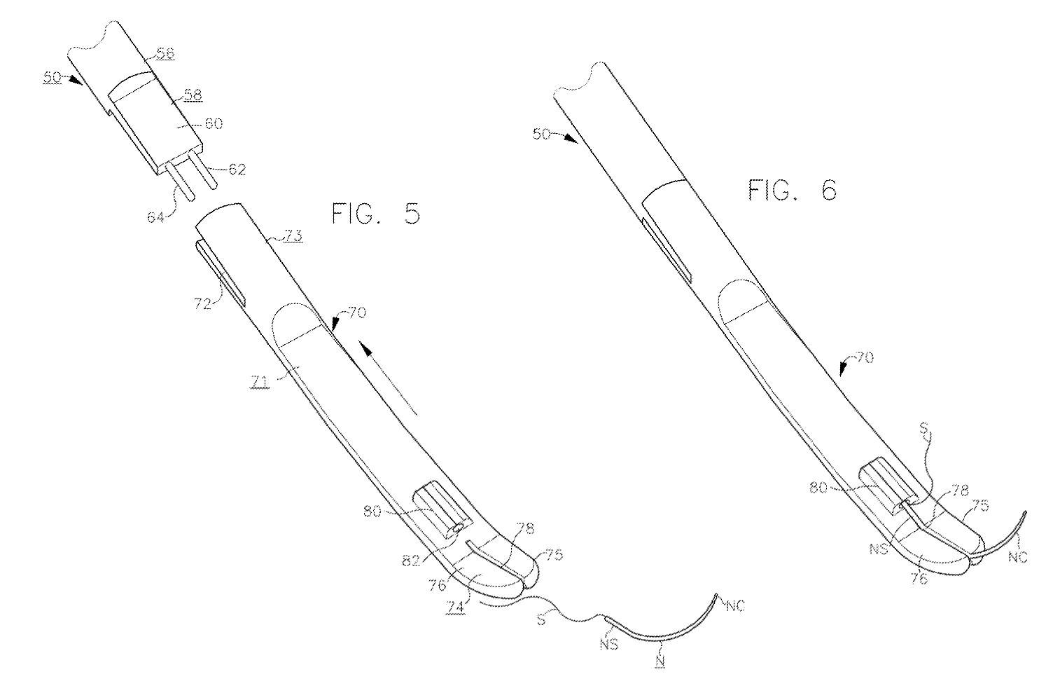

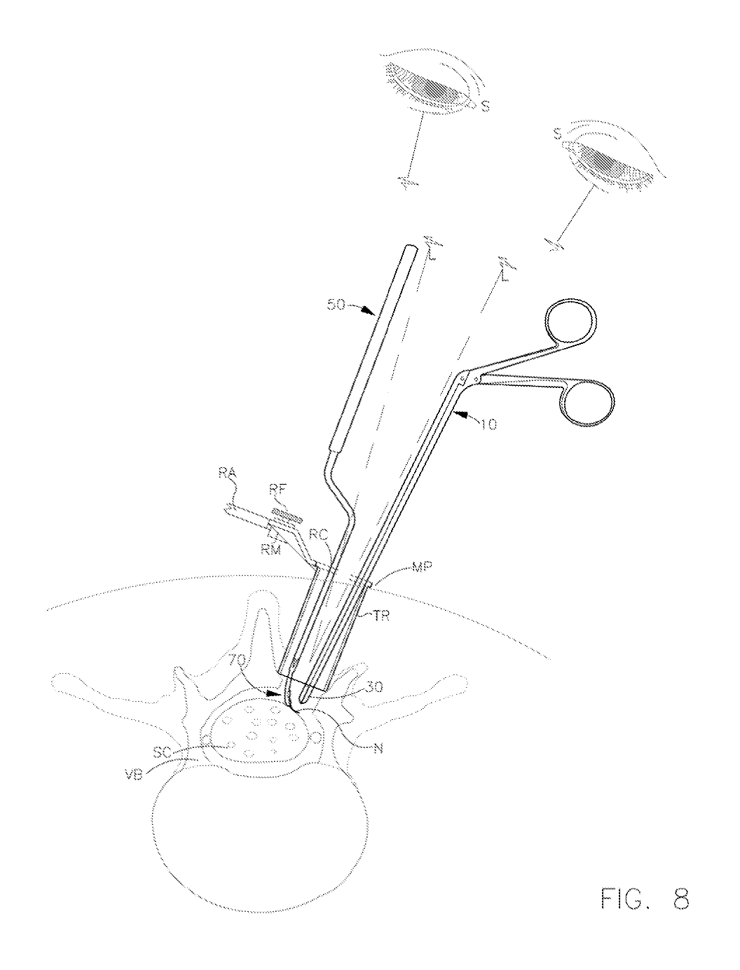

FIG. 7 illustrates another advantageous application of the aspect of the invention resulting from the use of a removable needle positioning tip. FIG. 7 depicts a needle cassette 90 that holds a number of needle assemblies 92, each of which includes a sterilized positioning tip 70 with a sterile premounted needle N and its attached suture S. The needle assemblies 92 will be held in the cassette 90 by dividers between individual assemblies (not shown) in any other suitable fashion that makes them readily removable from the cassette 90 by a surgeon during a surgical procedure. Each needle assembly can be used for an individual suture and then discarded and replaced for the next suture. The full advantages of this arrangement will be more apparent in the description below of a surgical procedure in accordance with method aspects of the invention.

The needle positioning tip 70 can be made of any suitable material, an example being stainless steel for its ease of sterilization. However, more preferably it will be a suitable plastic material for a number of reasons. One is that the fingers 75 and 76 will be more easily deformed to accept the needle and hold it in place, and will also release it more readily when desired. Another reason. is that the disposable needle tip assemblies 92 will be less expensive. Still another is that a suitable plastic material will permit the needle positioning tip 70 to be more bent into different shapes to facilitate the surgical procedures described herein. That is, the topography of surgical sites can vary and while a needle holder 50 using a fixed-configuration positioning tip 70 will still be superior to known surgical techniques using prior art instruments, the utility of the positioning tip can be enhanced if the surgeon can bend it to account for particular surgical site topographies.

It should be understood that using a removable needle positioning tip is optional, and that the needle holder 50 and needle positioning tip 70 can be provided as an integral unit. In addition, the same integral unit can be provided with a needle N and suture S in place as shown in FIG. 6, presterilized and separately packaged. The needle holder/positioning tip unit can be made of plastic with the intent that it be discarded after the premounted needle and suture have been used for suturing. Alternatively, the unit can be a more durable material, such as stainless steel, which after use can be returned to the manufacturer or other location for resterilization, remounting a needle and suture, and repackaging.

### Minimally Invasive Spinal Dural Procedures Using the Instruments

FIG. 8 schematically illustrates a surgical site that has been prepared for a spinal dural procedure using the minimally invasive technique described in the Medtronic brochure and U.S. Pat. No. 5,792,044. As described in those references, a tubular retractor TR has been positioned between vertebrae VB of the patient with its distal end proximate to but typically spaced from the patient's spinal cord SC. The proximal end of the retractor extends to a location external of the patient to provide access to the surgical site at the tube's distal end. As already discussed, the surgical site is prepared to provide the arrangement shown in FIG. 8 by first inserting a guidewire into a small incision at a medial posterior approach MP to the patient's spine. The surgeon will position the end of the guidewire at the desired location proximate to (but usually not touching) the patient's dura where the surgical procedure is to take place. After a series of larger and larger telescoping tissue dilators has provided a suitable opening, the tubular retractor TR is placed over the dilators, which are then removed, leaving the retractor TR in place as shown in FIG. 8.

FIG. 8A is a detailed view of the surgical retractor TR and is included for ease of reference. As already noted, it is a conventional part and is available in numerous sizes from various sources, including Medtronic Sofamor Danek USA, Inc., 800 Pyramid Place, Memphis, Tenn. 38132. The tubular retractor TR is molded in one piece from a transparent plastic material, with a cannula RC extending the length of the retractor's generally tubular body RB and a mounting bracket RM extending transversely from the body at its proximal end. However, retractors suitable for the purpose can also be made of other materials, such as stainless steel. The retractor body is depicted in FIG. 8A as being slightly tapered from proximal end to distal end, which facilitates insertion into the proper location within the patient. In other depictions herein the retractor is shown without the taper, and both configurations are suitable for the purpose. After the retractor TR is in position, the bracket RM is secured to one end of an anchoring arm RA by a suitable fastener RF, and the other end of the anchoring arm is secured to surrounding structure such as the surgical table (not shown). A light source, including one or more of a fiber optic cable or an LED on the end of a narrow rod inserted into the tubular retractor along an internal wall of the cannula to illuminate the surgical field, a headlamp worn by the surgeon, or an operating microscope with illumination that is used to magnify the surgical site. The drawing omits a light source for the sake of clarity.

Referring back to FIG. 8, it illustrates the unobstructed line of sight L from the surgeon's eyes S to the surgical field at the distal end of the tubular retractor TR when the surgeon uses the pistol-grip forceps 10 with one hand and the bayoneted needle holder 50 and needle positioning tip 70 with the other hand. It will be understood that the line of sight L depicted in FIG. 8 can also represent an operating loupe visualization or the line of sight provided by an operating microscope. The curved distal end of the needle positioning tip 70, oriented as discussed above, positions the needle more toward the center of the retractor tube to give the surgeon an even better view for suturing. It will be appreciated from FIG. 8 that a minimally invasive surgical technique using instruments according to the present invention is greatly facilitated as compared to instruments available to the prior art. Making such procedures more widely available will improve patient outcomes because minimally invasive surgery in almost all instances reduces postoperative discomfort to the patient and shortens recovery and rehabilitation times.

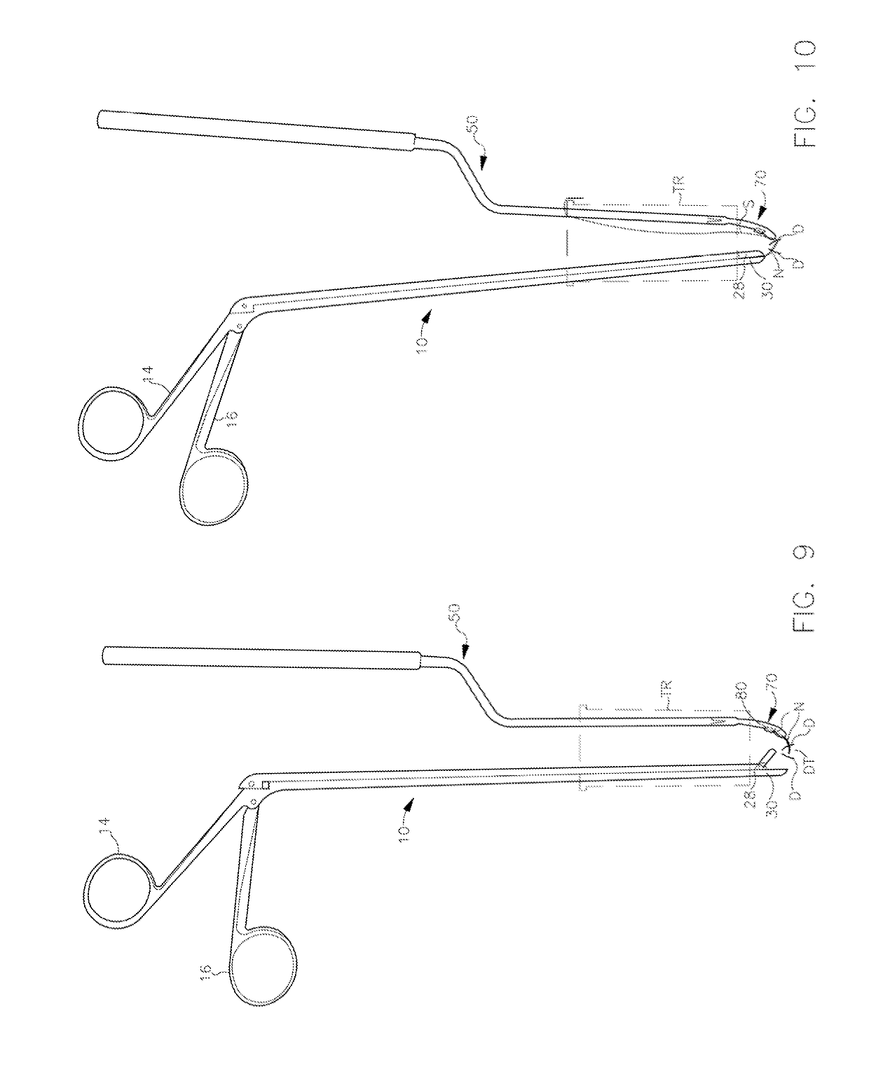

FIGS. 9 to 11 are highly schematic illustrations of steps in a method of using the forceps 10 and the needle holder 50 after the surgical site is prepared and the instruments are in place as shown in FIG. 8. Reference is also made to FIGS. 10 to 12 of the '024 application and their accompanying text. FIGS. 9 to 11 illustrate and describe essentially the same procedure described in '024 application, except the procedure depicted in FIGS. 9 to 11 use the instruments described therein. Beginning with FIG. 9, the surgeon uses the needle holder 50, with the mounted needle positioning tip 70 holding a needle N, as a needle pushing instrument to push the needle N through an edge of a tear DT in the dura D until the needle tip emerges on the other side of the tear. FIG. 10 shows the forceps 10, with the movable jaw 28 dosed and the distal portion of the needle N held between the jaws 28 and 30, used as a needle driving instrument to pull the needle completely through the dura D. As the needle N is driven through the dura using the forceps, the surgeon also manipulates the forceps and the needle holder to extract the needle from the needle slot 78 and the needle receptacle 80 of the needle positioning tip 70 (see FIGS. 5 and 6). FIG. 11 shows the needle N and suture S having been driven completely through the dura, and the needle holder 50 withdrawn from the retractor TR. The surgeon can then complete the suturing in a conventional fashion, such as by performing the “throw” of the knot outside of the patient and then sliding it down the retractor cannula with the appropriate instruments to tie together the edges of the dura at the tear DT. FIG. 11 shows that the needle N has been removed from the needle positioning tip 70, which in a preferred embodiment can be removed from the needle holder and discarded. If additional sutures are to be made, the surgeon can mount onto the needle holder 10 another sterile needle assembly 92 as described above in connection with FIG. 7.

Those skilled in the art will recognize several advantages that accrue through the use of the instruments described herein for spinal dural procedures. For one, the instruments, like their counterparts in the '024 application, afford the surgeon a clear view of the surgical field and the needle holder enables the surgeon to orient, the needle for the optimum path through the dural disruption. In addition, a curved needle positioning tip according to the described embodiment of the present invention provides an even clearer view of the surgical field. And if the edge of the dura to be sutured is folded or otherwise awkwardly disposed, the curved distal portion of the needle positioning tip permits the surgeon to press on the dura with the tip of the needle still facing the dura edge and in position to be pushed through it. The smooth, blunt end of the needle positioning tip enables the spinal dura to be gently pushed in this fashion without tearing it. Further, making the needle positioning tip from a material that the surgeon can bend into different shapes permits even more options as to orienting the needle for the optimum path through the dura.

### SUMMARY AND CONCLUSION

The above description uses as exemplary embodiments of the invention surgical instruments and a surgical procedure for repairing spinal dural tears and disruptions. Those skilled in the art will appreciate that the invention is not so limited, and has general applicability to surgical procedures at locations and on tissues and organs other than the spinal dura. In that same connection, those skilled in the art will readily recognize that only selected preferred embodiments of the invention have been depicted and described, and it will be understood that various changes, modifications, uses, and applications of the devices and methods described herein can be made other than those specifically mentioned above without departing from the spirit and scope of the invention, which is defined solely by the claims that follow.

1. A needle driving instrument for grasping a surgical needle at a surgical site accessible through a cannula extending from a proximal end to a distal end at the surgical site, the instrument comprising:
a pair of handles pivotally mounted relative to each other, wherein the handles include grips for enabling a surgeon to pivot one handle relative to the other using one hand;
a shaft portion to which the handles are mounted, wherein the handles extend laterally from a proximal portion of the shaft portion for providing a line of sight enabling the surgeon to view the surgical site when holding the grips while the shaft portion is within the cannula and the distal end is proximate to the surgical site; and
a pair of jaws mounted at a distal end of the shaft portion for movement between an open position and a closed position, wherein facing surfaces of the jaws are forced together into the closed position for grasping the needle when one handle is pivoted relative to the other, the facing surfaces having a surface for frictionally engaging the needle with sufficient force to enable the surgeon to drive the needle through tissue at the surgical site without damaging the needle.

2. The instrument in claim 1, wherein the facing surfaces of the jaws have one of a knurled texture and beaded texture comprising raised protuberances with blunt ends.

3. The instrument in claim 2, wherein the raised protuberances are spaced apart a distance of about the same order of magnitude as the diameter of the needle.

4. The instrument in claim 3, wherein the raised protuberances are spaced between 0.5 mm and 1.0 mm apart.

5. A needle pushing instrument for holding a surgical needle at a surgical site accessible through a cannula extending from a proximal end to a distal end at the surgical site, the instrument comprising:
a needle holder comprising a distal portion connected by an intermediate portion to a proximal handle portion, wherein the handle portion has an axis offset laterally from an axis of the distal portion to form a bayoneted instrument for providing a line of sight enabling the surgeon to view the surgical site while holding the handle portion while the distal portion is within the cannula and the distal end of the holder is proximate to the surgical site; and
a needle positioning tip having a proximal end secured to the distal end of the needle holder, the needle positioning tip having a needle slot extending from the distal end toward the proximal end and a needle receptacle on a main portion between the proximal and distal ends and aligned with the slot, wherein the slot removably captures a shaft portion of the needle therein and the receptacle removably holds a shank end portion of the needle.

6. The instrument in claim 5, wherein the needle holder and the needle positioning tip are a single unitary part.

7. The instrument in claim 5, wherein the needle holder is a single unitary part and the needle positioning tip is removably secured to the needle holder.

8. The instrument in claim 7, further comprising positioning means for positioning the needle positioning tip in a predetermined orientation on the needle holder.

9. The instrument in claim 8, wherein the positioning means includes a positioning slot in the proximal end of the needle positioning tip and an indexing tab at the distal end of the needle holder, the indexing tab being constructed for insertion into the positioning slot to orient the needle positioning tip on the needle holder in a predetermined orientation.

10. The instrument in claim 9, wherein the positioning means further includes at least two indexing pins extending from the distal end of the indexing tab for insertion into cooperating recesses at the base of the positioning slot.

11. The instrument in claim 8, wherein the needle positioning tip can be positioned in one of at least two predetermined orientations on the needle holder.

12. The instrument in claim 5, wherein the needle holder lies in a plane and the needle slot extends along a distal end portion of the needle positioning tip and opens at a slot in the distal end thereof, the distal portion of the needle positioning tip being curved to form a bifurcated tip portion that is curved in a direction away from the handle and in a plane perpendicular to the plane of the needle holder.

13. The instrument in claim 5 for use with a needle lying in a plane and having a straight shank end portion with a suture secured thereto and a curved shaft portion, wherein the needle slot is straight and the receptacle includes a hollow bore for accepting the needle shank portion with the suture extending from the bore in the same direction as the needle shank portion.

14. A method for repairing a disruption in the spinal dura, the method comprising:
exposing a surgical site proximate to the spinal dura using a tubular retractor with a distal end proximate to the spinal dura, a proximal end, and a cannula connecting the distal and proximal ends; providing a needle driving instrument having (i) a pair of handles pivotally mounted relative to each other, wherein the handles include grips for enabling a surgeon to pivot one handle relative to the other using one hand, (ii) a shaft portion to which the handles are mounted, wherein the handles extend laterally from a proximal portion of the shaft portion for providing a line of sight enabling the surgeon to view the surgical site when holding the grips while the shaft portion is within the cannula and the distal end is proximate to the surgical site, and (iii) a pair of jaws mounted at a distal end of the shaft portion for movement between an open position and a closed position, wherein facing surfaces of the jaws are forced together into the closed position for grasping a needle when one handle is pivoted relative to the other, the facing surfaces having a surface for frictionally engaging the needle with sufficient force to enable the surgeon to drive the needle through tissue at the surgical site without damaging the needle;
a needle pushing instrument having (i) a needle holder comprising a distal portion connected by an intermediate portion to a proximal handle portion, wherein the handle portion has an axis offset laterally from an axis of the distal portion to form a bayoneted instrument for providing a line of sight enabling the surgeon to view the surgical site while holding the handle portion with the other hand while the distal portion is within the cannula and the distal end of the holder is proximate to the surgical site, (ii) a needle positioning tip having a proximal end secured to the distal end of the needle holder, and (iii) a surgical needle removably held by the needle positioning tip;
pushing the needle shaft portion through the dura from one side of the disruption through the dura on another side of the disruption using the needle pushing instrument;
grasping the needle shaft portion on the other side of the disruption between the jaws of needle driving instrument;
manipulating the needle pushing instrument and the needle driving instrument to remove the needle from the needle positioning tip;
driving the needle and the suture through the edges of the dura using the needle driving instrument; and
tying off the suture to bring the edges of the dura together.

15. The method in claim 14, wherein the needle positioning tip is removably mounted to the needle holder, the method further comprising removing the needle positioning tip after the driving step, mounting another needle driving tip to the needle holder, and thereafter repeating the pushing grasping, manipulating, driving, and tying off steps.

16. The method in claim 15, wherein the needle positioning tip further comprises positioning means for positioning the needle positioning tip in a predetermined orientation on the needle holder.

17. The method in claim 16, wherein the positioning means includes a positioning slot in the proximal end of the needle positioning tip and an indexing tab at the distal end of the needle holder, the indexing tab being constructed for insertion into the positioning slot to orient the needle positioning tip on the needle holder in a predetermined orientation.

18. The method in claim 14, wherein the needle has a shaft portion and a shank end portion with a suture attached thereto, wherein the needle shaft portion is removably captured in a needle slot extending from a distal end toward a proximal end of the needle positioning tip, and the needle shank portion and the suture are removably held in a needle receptacle on a main portion of the needle positioning tip between its proximal and distal ends, and the manipulating step includes removing the needle shaft portion from the needle slot and the needle shank portion and suture from the needle receptacle.

19. The method in claim 18, wherein the needle positioning tip is removably mounted to the needle holder, the method further comprising removing the needle positioning tip after the driving step, mounting another needle driving tip to the needle holder, and thereafter repeating the pushing, grasping, manipulating, and driving steps.

20. The method in claim 14, wherein the cannula is between about 14 mm and 26 mm in diameter and between about 40 mm and 120 mm long.
